(19) United States
(12) Patent Application Publication
(10) Pub. No.: US 2025/0165683 A1
(43) Pub. Date: May 22, 2025

(54) ARTIFICIAL INTELLIGENCE MODEL-BASED COMPUTING DEVICE AND ANALYSIS METHOD FOR OPTIMAL DESIGN OF SECONDARY BATTERY ELECTROLYTE

(71) Applicant: Korea Electronics Technology Institute, Seongnam-si (KR)

(72) Inventors: Jaekyu Lee, Yongin-si (KR); Sang Yub Lee, Yongin-si (KR); Inpyo Cho, Yongin-si (KR)

(21) Appl. No.: 18/946,547

(22) Filed: Nov. 13, 2024

(30) Foreign Application Priority Data
Nov. 16, 2023 (KR) 10-2023-0159608

(51) Int. Cl. G06F 30/28; G01R 31/367

(52) U.S. Cl. CPC G06F 30/28; G01R 31/367

(57) ABSTRACT

Proposed is an artificial intelligence (AI) model-based analysis method for an optimal design of a secondary battery electrolyte. The AI model-based analysis method may include obtaining a tomography image of a calendared battery material, and inputting the tomography image to a pre-trained AI model. The method may also include outputting a viscosity and a transmittance of a battery electrolyte through the AI model. The AI model may include a first AI model configured to analyze porosity distribution and tortuosity information about the tomography image input thereto. The AI model may also include a second AI model configured to output a viscosity and a transmittance corresponding to the electrolyte, based on the porosity distribution and tortuosity information.

### CROSS-REFERENCE TO RELATED APPLICATIONS

This application claims the benefit of the Korean Patent Application No. 10-2023-0159608 filed on Nov. 16, 2023, which is hereby incorporated by reference in its entirety.

### BACKGROUND

### Technical Field

The present disclosure relates to an artificial intelligence (AI) model-based computing device and analysis method for an optimal design of a secondary battery electrolyte.

### Description of Related Technology

An electrolyte of batteries, one of main elements of batteries, stores and transfers electrical energy and largely affects the performance and characteristic of batteries. Also, electrolytes enable ion movement between a positive electrode and a negative electrode, and this corresponds to the main operation principle of batteries. The reason that various kinds of batteries use various kinds of electrolytes is because the physical properties and characteristic of electrolytes are associated with the capacity, voltage, stability, charge/discharge characteristic, and lifetime of batteries.

### SUMMARY

One aspect is an artificial intelligence (AI) model-based computing device and analysis method for an optimal design of a secondary battery electrolyte, which may segment a porosity distribution of a tomography image through an AI model to calculate an electrolyte injection characteristic-based transmittance and a viscosity of an electrolyte, so as to deduce an optimal electrolyte material used in a secondary battery.

Aspects of the present disclosure are not limited to the aforesaid, but other aspects not described herein will be clearly understood by those skilled in the art from descriptions below.

Another aspect is an artificial intelligence (AI) model-based analysis method for an optimal design of a secondary battery electrolyte, the AI model-based analysis method including: a step of obtaining a tomography image of a calendared battery material; a step of inputting the tomography image to a pre-trained AI model; and a step of outputting a viscosity and a transmittance of a battery electrolyte through the AI model, wherein the steps are performed by a computer, and the AI model may include a first AI model configured to analyze porosity distribution and tortuosity information about the tomography image input thereto and a second AI model configured to output a viscosity and a transmittance corresponding to the electrolyte, based on the porosity distribution and tortuosity information.

In some embodiments of the present disclosure, the AI model-based analysis method may further include: a step of collecting tomography surface image data of a real battery; a step of generating simulation image data based on a virtual environment, based on the tomography surface image data of the real battery; and a step of training and generating the first AI model, based on the tomography surface image data of the real battery and the simulation image data.

In some embodiments of the present disclosure, the step of training and generating the first AI model may include: a step of configuring a backbone model based on at least one of 3D U-Net (U-Net) mask R-CNN and bilateral segmentation network (BiSeNet) segmentation models; and a step of performing re-training of the backbone model to generate the first AI model, based on a transfer learning environment.

In some embodiments of the present disclosure, the AI model-based analysis method may further include: a step of obtaining porosity distribution and tortuosity information output through the first AI model; a step of obtaining a plurality of simulated data through a computational fluid dynamics (CFD)-based simulation, based on the porosity distribution and tortuosity information; and a step of training and generating the second AI model, based on the plurality of simulated data.

Another aspect is an artificial intelligence (AI) model-based computing device for an optimal design of a secondary battery electrolyte, the AI model-based computing device including: a communication module configured to obtain a tomography image of a battery material; a memory configured to store a pre-trained AI model; and a processor configured to input the tomography image to an AI model through execution of a program stored in the memory to output a viscosity and a transmittance of a battery electrolyte, wherein the AI model may include a first AI model configured to analyze a porosity distribution and a tortuosity on the tomography image input thereto and a second AI model configured to output a viscosity and a transmittance corresponding to the electrolyte, based on the porosity distribution and the tortuosity.

In some embodiments of the present disclosure, the processor may collect tomography surface image data of a real battery by using the communication module, may generate simulation image data based on a virtual environment, based on the tomography surface image data of the real battery, and may train and generate the first AI model, based on the tomography surface image data of the real battery and the simulation image data.

In some embodiments of the present disclosure, the processor may configure a backbone model based on at least one of 3D U-Net (U-Net) mask R-CNN and bilateral segmentation network (BiSeNet) segmentation models and may perform re-training of the backbone model to generate the first AI model, based on a transfer learning environment.

In some embodiments of the present disclosure, the processor may obtain porosity distribution and tortuosity information output through the first AI model, may obtain a plurality of simulated data through a computational fluid dynamics (CFD)-based simulation, based on the porosity distribution and tortuosity information, and may train and generate the second AI model, based on the plurality of simulated data.

Furthermore, another method and another system for implementing the present disclosure and a computer-readable recording medium storing a computer program for executing the method may be further provided.

The present disclosure relates to an AI model and a use method thereof, which may be for calculating an electrolyte injection characteristic-based transmittance and a viscosity of an electrolyte by segmenting a porosity and a tortuosity, based on a tomography image of a material included in a secondary battery (cell), and thus, may realize the following effects.

First, in terms of performance prediction, a transmittance and a viscosity of an electrolyte may be calculated from one image, and the performance (capacity, voltage, stability, etc.) of a corresponding battery may be predicted by using calculated data.

Second, in terms of process analysis, the efficiency of a corresponding battery process (mixing and rolling) may be checked by quickly segmenting a structure of a battery material.

Third, in terms of performance enhancement, the calculation of a transmittance and a distribution of a battery electrolyte (additives) which is very large in computing load may be considerably reduced by using a computational fluid dynamics (CFD)-based pre-simulation and AI technology.

As described above, according to an embodiment of the present disclosure, the performance of a battery may be more quickly, conveniently, and accurately analyzed than an operation of checking the efficiency of the battery and a battery process, based on conventional X-ray tomography photograph.

It is to be understood that both the foregoing general description and the following detailed description of the present disclosure are exemplary and explanatory and are intended to provide further explanation of the invention as claimed.

### BRIEF DESCRIPTION OF THE DRAWINGS

The accompanying drawings, which are included to provide a further understanding of the disclosure and are incorporated in and constitute a part of this application, illustrate embodiments of the disclosure and together with the description serve to explain the principle of the disclosure.

FIG. 1 is a flowchart of an artificial intelligence (AI) model-based analysis method according to an embodiment of the present disclosure.

FIG. 2 is a diagram illustrating a manufacturing process of a battery.

FIG. 3 is a diagram for describing a first AI model in an embodiment of the present disclosure.

FIG. 4 is a diagram illustrating a transfer learning environment for generating a first AI model in an embodiment of the present disclosure.

FIG. 5 is a diagram for describing first and second AI models in an embodiment of the present disclosure.

FIG. 6 is a diagram illustrating a composite tomography surface and efficiency of a real nickel cobalt manganese (NCM) battery.

FIG. 7 is a block diagram of an AI model-based computing device according to an embodiment of the present disclosure.

### DETAILED DESCRIPTION

Various manufacturing processes are performed for manufacturing batteries, and particularly, nickel cobalt manganese (NCM) batteries undergo mixing, kneading, and compression processes of each raw material. An electrolyte to be used in a corresponding battery is manufactured in a material obtained through mixing and compression processes. Electrolytes of batteries are generally manufactured by using lithium salt, and this should maintain a very precise concentration and properties.

The advantages, features and aspects of the present disclosure will become apparent from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art.

The terms used herein are for the purpose of describing particular embodiments only and are not intended to be limiting of example embodiments. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Herein, like reference numeral refers to like element, and “and/or” include(s) one or more combinations and each of described elements. Although “first” and “second” are used for describing various elements, but the elements are not limited by the terms. Such terms are used for distinguishing one element from another element. Therefore, a first element described below may be a second element within the technical scope of the present disclosure.

Unless otherwise defined, all terms (including technical and scientific terms) used herein may be used as a meaning capable of being commonly understood by one of ordinary skill in the art. Also, terms defined in dictionaries used generally are not ideally or excessively construed unless clearly and specially defined.

The present disclosure relates to an artificial intelligence (AI) model-based computing device and analysis method for an optimal design of a secondary battery electrolyte.

An embodiment of the present disclosure may be for training, generating, and applying an AI model for an optimal design of a secondary battery electrolyte of a micro structure level, may segment and analyze a porosity distribution of a tomography image by using deep learning, and may use information, obtained based thereon, in determining a space path and an internal structure of an electrolyte.

Moreover, a viscosity and a transmittance of an electrolyte may be estimated by using simulation data, and thus, the performance of the electrolyte may be predicted, and an optimal electrolyte material may be developed and manufactured. An electrolyte developed based thereon may be optimized based on a viscosity and a transmittance of the electrolyte and may enhance the performance and stability of a battery.

On the other hand, an embodiment of the present disclosure may be applied for deducing a manufacturing process of a battery and a porosity distribution, which may optimize the performance of the battery and an electrolyte.

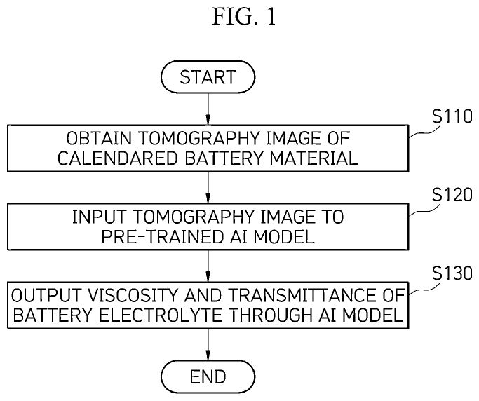

Hereinafter, an AI model-based analysis method for an optimal design of a secondary battery electrolyte, performed by a computing device 100 according to an embodiment of the present disclosure, will be described with reference to FIGS. 1 to 6.

FIG. 1 is a flowchart of an AI model-based analysis method according to an embodiment of the present disclosure.

The AI model-based analysis method according to an embodiment of the present disclosure may include a step S110 of obtaining a tomography image of a calendared battery material, a step S120 of inputting (or feeding) the tomography image to a pre-trained AI model, and a step S130 of outputting a viscosity and a transmittance of a battery electrolyte through the AI model.

It may be understood that each step illustrated in FIG. 1 is performed by the computing device 100 described below, but the present disclosure is not limited thereto.

First, an embodiment of the present disclosure may obtain a tomography image of the calendared battery material in step S110.

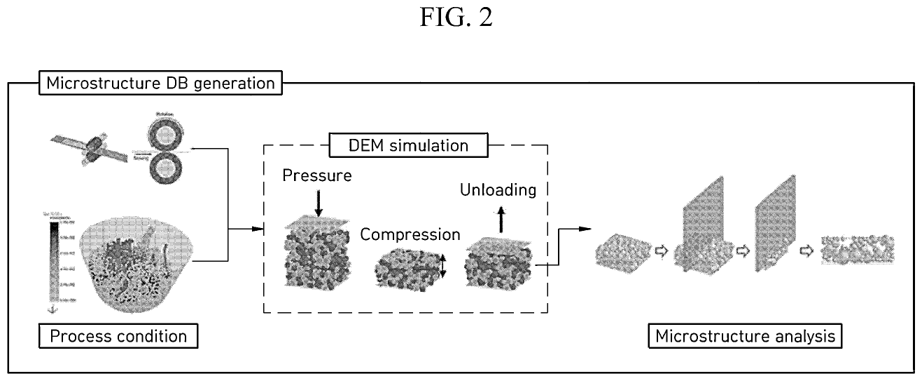

To manufacture a secondary battery (cell), a process such as a material manufacturing process, an electrolyte manufacturing process, an electrode manufacturing process, a cell assembly process, and a battery packaging process should be performed from a process of collecting raw materials (for example, nickel cobalt manganese (NCM)/nickel, cobalt, maganese, etc.). FIG. 2 illustrates a process of forming a material configuring an inner portion of a battery pack through mixing and calendaring of raw materials.

In such a process, an embodiment of the present disclosure may be based on a tomography image of a calendared battery material. In this case, calendaring may be a process of compressing an electrolyte and an active material of a battery to be flat and have a certain thickness, and a battery cell may be formed in a consistent structure by adjusting a thickness and a height of each of a separator and an electrode of the battery.

Subsequently, an embodiment of the present disclosure may input the tomography image to a pre-trained AI model in step S120 and may output a viscosity and a transmittance of a battery electrolyte through the AI model in step S130. That is, an embodiment of the present disclosure may calculate the viscosity and transmittance of the electrolyte affecting the performance of the battery to previously determine the performance of the battery.

In this case, the AI model applied to an embodiment of the present disclosure may include a first AI model which analyzes porosity distribution and tortuosity information about the tomography image input thereto and a second AI model which outputs a viscosity and a transmittance corresponding to the electrolyte, based on the porosity distribution and tortuosity information.

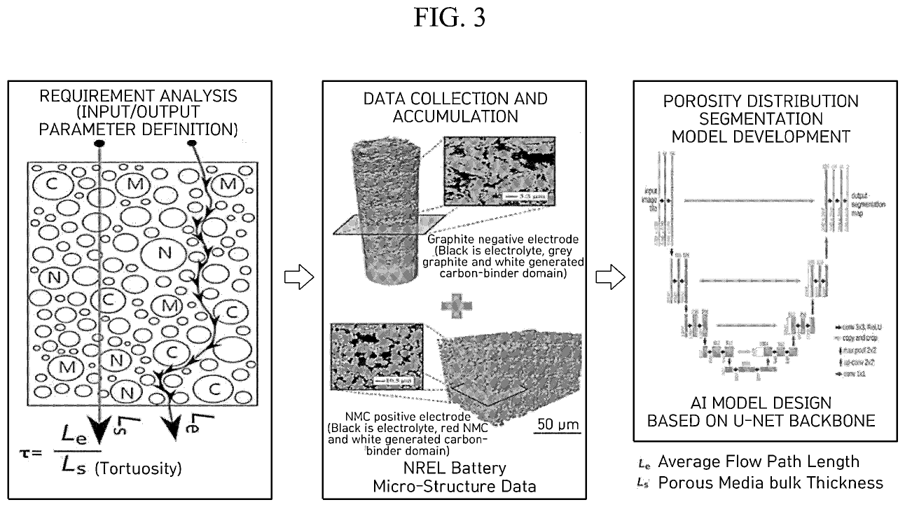

FIG. 3 is a diagram for describing a first AI model in an embodiment of the present disclosure. In this case, FIG. 3 (a) illustrates a concept of a tortuosity and a porosity distribution which is a distribution of battery raw materials, FIG. 3 (b) illustrates an example of collected and accumulated data, and FIG. 3 (c) illustrates an example of a process of generating the first AI model.

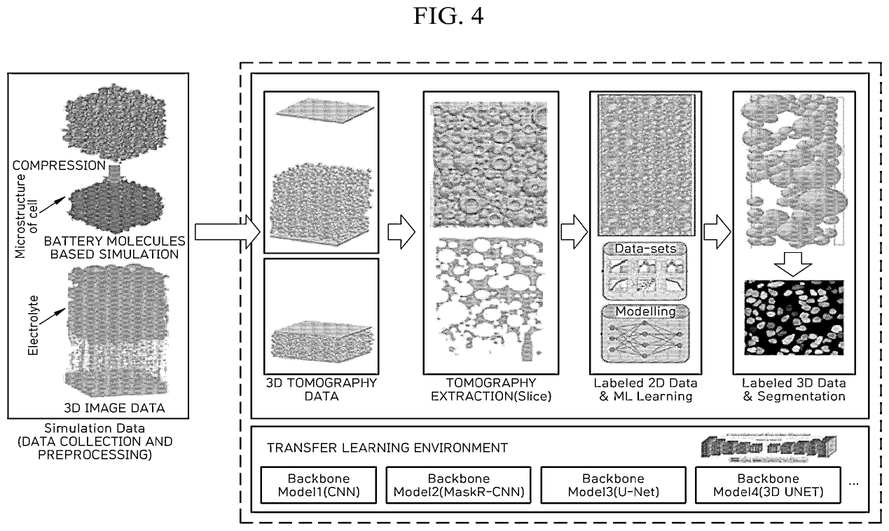

FIG. 4 is a diagram illustrating a transfer learning environment for generating a first AI model in an embodiment of the present disclosure.

In an embodiment, the first AI model may be a segmentation model based on a three-dimensional (3D) image data set and may be trained by using transfer learning.

In this case, the 3D image data set for training of the first AI model for outputting a porosity distribution and a tortuosity may use simulation image data based on a virtual environment instead of using a real battery tomography image. That is, an embodiment of the present disclosure may generate simulation image data based on a virtual environment, based on real battery tomography image data. The simulation image data may be synthetic data generated in various virtual environments, based on a mathematic model and computer graphics technology.

The synthetic data may denote virtual data generated based on real data, and when the real data is insufficient because data may be extracted in various environments, the synthetic data may be used. Also, data may be freely augmented (rotate, extend, noise addition, etc.), and thus, various data sets may be generated, and a test based on a virtual environment may be performed.

Furthermore, the first AI model for training of the first AI model may be data which is generated by using a discrete element modeling technique. For example, micro-particles of elements (an active material, a separator, an electrolyte, etc.) of a battery may be represented by discrete units, and by simulating an interaction, a collision, and a motion between the micro-particles, simulation image data may be generated based on a simulation result of mixing and arrangement of various materials.

Subsequently, the first AI model which is a segmentation AI model may be trained and generated based on simulation image data based on a virtual environment along with tomography image data of a battery. In this case, training of the first AI model may configure a backbone model based on at least one of 3D U-Net (U-Net) mask R-CNN and bilateral segmentation network (BiSeNet) segmentation models, and the backbone model may perform a re-training process, based on a transfer learning environment, thereby implementing an optimal first AI model.

As described above, an embodiment of the present disclosure may perform a process of collecting and preprocessing simulation-based 3D image data (volumetric images) so as to highly enhance a porosity distribution analysis of a battery tomography image, and by applying such a process, the first AI model which is a deep learning AI image segmentation model for porosity distribution segmentation may be generated.

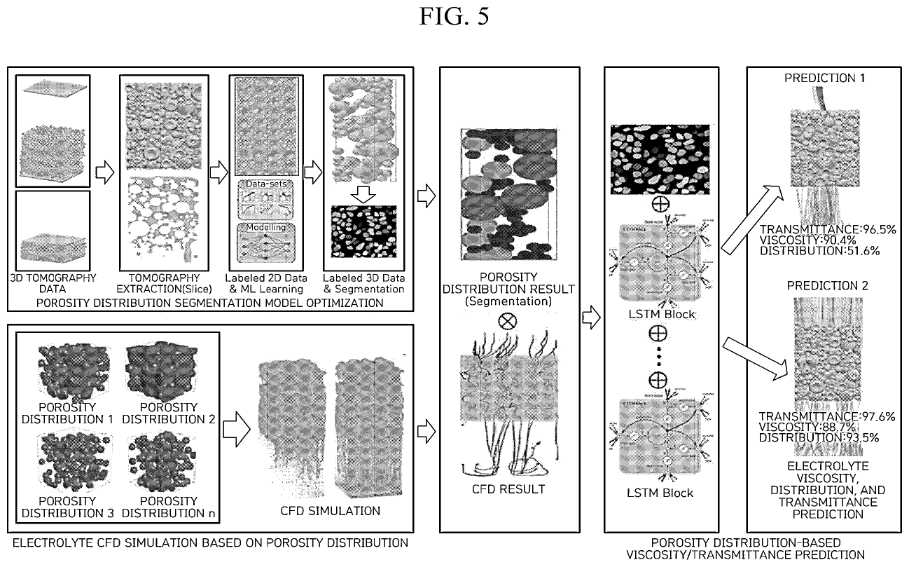

FIG. 5 is a diagram for describing first and second AI models in an embodiment of the present disclosure.

An embodiment of the present disclosure, as described above, may be characterized in that two AI models are configured as a composite AI model. In this case, the first AI model may be an AI model which performs image segmentation for analyzing porosity distribution and tortuosity information, and the second AI model may be an AI model which predicts a viscosity and a transmittance of an electrolyte, based on the porosity distribution and tortuosity information which is an output result of the first AI model.

In an embodiment, a training process of the second AI model may first obtain the porosity distribution and tortuosity information output through the first AI model and may obtain a plurality of simulated data through a computational fluid dynamics (CFD)-based simulation, based on the porosity distribution and tortuosity information. Subsequently, the second AI model may be trained and generated based on the plurality of simulated data.

In this case, data used in training of the second AI model may be data which is obtained through a plurality of CFD-based simulations, based on a result calculated by a discrete element modeling technique. That is, virtual learning data may be generated by modeling and simulating a physical phenomenon (a temperature distribution and the other characteristic) associated with a viscosity and a flow of an electrolyte in a battery.

When the first and second AI models are trained and generated through such a process, the performance (efficiency) of a battery and a process may be checked based on only an actually mixed battery tomography surface (based on X-ray) by using a calculated AI model.

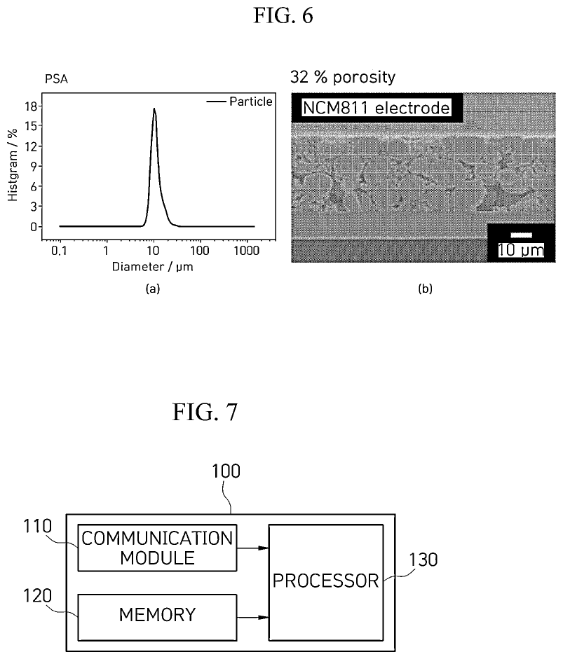

FIG. 6 is a diagram illustrating a composite tomography surface (left) and efficiency (right) of an NCM battery. Fundamentally, in a case where a battery raw material composite tomography surface is configured with particles having a uniform size as in FIG. 6, performance (efficiency) may be good.

FIG. 7 is a diagram illustrating porosity distribution segmentation based on a battery tomography surface image. An embodiment of the present disclosure may analyze a particle structure and distribution, based on a porosity distribution segmentation model (a first AI model or an AI model including the same), and thus, may calculate an optimal electrolyte material.

In the above description, steps S110 to S130 may be further divided into additional steps, or may be combined into fewer steps. Also, depending on the case, some steps may be omitted, and the order of steps may be changed. Despite other omitted descriptions, descriptions given with reference to FIGS. 1 to 6 may be applied to descriptions of the computing device 100 of FIG. 7.

FIG. 7 is a block diagram of an AI model-based computing device 100 according to an embodiment of the present disclosure.

The computing device 100 according to an embodiment of the present disclosure may include a communication module 110, a memory 120, and a processor 130.

The communication module 110 may obtain a tomography image of a battery material. The communication module 110 may include a wired communication module and a wireless communication module. The wired communication module may be implemented with a power line communication device, a telephone wire communication device, a cable home (MoCA), Ethernet, IEEE1294, an integration wired home network, an RS-485 control device, and/or the like. Also, the wireless communication module may be implemented with wireless local area network (WLAN), Bluetooth, HDR WPAN, UWB, ZigBee, Impulse Radio, 60 GHz WPAN, Binary-CDMA, wireless USB technology, wireless HDMI technology, and/or the like.

A pre-trained AI model may be stored in the memory 120, and the processor 130 may execute a program stored in the memory 120. Here, the memory 120 may denote a non-volatile memory, which continuously retains information stored therein even when power is not supplied thereto, and a volatile memory.

In this case, the AI model may include a first AI model which analyzes a porosity distribution and a tortuosity on a tomography image input thereto and a second AI model which outputs a viscosity and a transmittance corresponding to the electrolyte, based on the porosity distribution and the tortuosity.

Examples of the memory 120 may include NAND flash memory such as compact flash (CF) card, secure digital (SD) card, memory stick, solid state drive (SSD), and micro SD card, magnetic computer memory device such as hard disk drive (HDD), and optical disc drive such as CD-ROM and DVD-ROM.

The processor 130 may execute a program stored in the memory 120, and thus, may input a tomography image to an AI model to output a viscosity and a transmittance of a battery electrolyte.

The AI model-based analysis method according to an embodiment of the present disclosure may be implemented as a program (or an application) and may be stored in a medium, so as to be executed in connection with a server which is hardware.

The above-described program may include a code encoded as a computer language such as C, C++, JAVA, or machine language readable by a processor (CPU) of a computer through a device interface of the computer, so that the computer reads the program and executes the methods implemented as the program. Such a code may include a functional code associated with a function defining functions needed for executing the methods, and moreover, may include an execution procedure-related control code needed for executing the functions by using the processor of the computer on the basis of a predetermined procedure. Also, the code may further include additional information, needed for executing the functions by using the processor of the computer, or a memory reference-related code corresponding to a location (an address) of an internal or external memory of the computer, which is to be referred to by a media. Also, when the processor needs communication with a remote computer or server so as to execute the functions, the code may further include a communication-related code corresponding to a communication scheme needed for communication with the remote computer or server and information or a media to be transmitted or received in performing communication, by using a communication module of the computer.

The stored medium may denote a device-readable medium semi-permanently storing data, instead of a medium storing data for a short moment like a register, a cache, and a memory. In detail, examples of the stored medium may include read only memory (ROM), random access memory (RAM), CD-ROM, a magnetic tape, floppy disk, and an optical data storage device, but are not limited thereto. That is, the program may be stored in various recording mediums of various servers accessible by the computer or various recording mediums of the computer of a user. Also, the medium may be distributed to computer systems connected to one another over a network and may store a code readable by a computer in a distributed scheme.

Operations of an algorithm or a method described above according to the embodiments of the present disclosure may be directly implemented as hardware, implemented as a software module executed by hardware, or implemented by a combination thereof. The software module may be provided in RAM, ROM, erasable programmable read only memory (EPROM), electrical erasable programmable read only memory (EEPROM), flash memory, a hard disk, an attachable/detachable disk, and CD-ROM, or a computer-readable recording medium of an arbitrary type well known to those skilled in the art.

It will be apparent to those skilled in the art that various modifications and variations can be made in the present disclosure without departing from the spirit or scope of the inventions. Thus, it is intended that the present disclosure covers the modifications and variations of this invention provided they come within the scope of the appended claims and their equivalents.

What is claimed is:

1. An artificial intelligence (AI) model-based analysis method for an optimal design of a secondary battery electrolyte, the AI model-based analysis method comprising:
obtaining a tomography image of a calendared battery material;
feeding the tomography image to a pre-trained AI model; and
outputting a viscosity and a transmittance of a battery electrolyte through the AI model,
the AI model comprising:
a first AI model configured to analyze porosity distribution and tortuosity information about the tomography image input thereto, and
a second AI model configured to output a viscosity and a transmittance corresponding to the electrolyte, based on the porosity distribution and tortuosity information.

2. The AI model-based analysis method of claim 1, further comprising:
collecting tomography surface image data of a real battery;
generating simulation image data based on a virtual environment, based on the tomography surface image data of the real battery; and
training and generating the first AI model, based on the tomography surface image data of the real battery and the simulation image data.

3. The AI model-based analysis method of claim 2, wherein the training and generating comprises:
configuring a backbone model based on at least one of 3D U-Net (U-Net) mask R-CNN and bilateral segmentation network (BiSeNet) segmentation models; and
performing re-training of the backbone model to generate the first AI model, based on a transfer learning environment.

4. The AI model-based analysis method of claim 1, further comprising:
obtaining porosity distribution and tortuosity information output through the first AI model;
obtaining a plurality of simulated data through a computational fluid dynamics (CFD)-based simulation, based on the porosity distribution and tortuosity information; and
training and generating the second AI model, based on the plurality of simulated data.

5. An artificial intelligence (AI) model-based computing device for an optimal design of a secondary battery electrolyte, the AI model-based computing device comprising:
a communication module configured to obtain a tomography image of a battery material;
a memory configured to store a pre-trained AI model; and
a processor configured to feed the tomography image to an AI model through execution of a program stored in the memory to output a viscosity and a transmittance of a battery electrolyte,
the AI model comprising:
a first AI model configured to analyze a porosity distribution and a tortuosity on the tomography image input thereto, and
a second AI model configured to output a viscosity and a transmittance corresponding to the electrolyte, based on the porosity distribution and the tortuosity.

6. The AI model-based computing device of claim 5, wherein the processor is configured to:
collect tomography surface image data of a real battery by using the communication module;
generate simulation image data based on a virtual environment, based on the tomography surface image data of the real battery, and trains; and
generate the first AI model, based on the tomography surface image data of the real battery and the simulation image data.

7. The AI model-based computing device of claim 6, wherein the processor is adapted to configure a backbone model based on at least one of 3D U-Net (U-Net) mask R-CNN and bilateral segmentation network (BiSeNet) segmentation models and perform re-training of the backbone model to generate the first AI model, based on a transfer learning environment.

8. The AI model-based computing device of claim 5, wherein the processor is configured to:
obtain porosity distribution and tortuosity information output through the first AI model;
obtain a plurality of simulated data through a computational fluid dynamics (CFD)-based simulation, based on the porosity distribution and tortuosity information, and trains; and
generate the second AI model, based on the plurality of simulated data.
